(19) United States
(12) Patent Application Publication
(10) Pub. No.: US 2026/0054086 A1
(43) Pub. Date: Feb. 26, 2026

(54) DEVICES AND METHODS FOR TREATMENTS USING MICROWAVE ENERGY

(71) Applicant: Emblation Limited, Alloa (GB)

(72) Inventors: Eamon McErlean, Alloa (GB); Gary Beale, Dunblane (GB); Matthew Donald Kidd, Stirling (GB)

(21) Appl. No.: 19/170,409

(22) Filed: Apr. 4, 2025

Related U.S. Application Data
(63) Continuation of application No. 16/869,886, filed on May 8, 2020, now abandoned.
(60) Provisional application No. 62/846,071, filed on May 10, 2019.

(51) Int. Cl. A61N 5/02

(52) U.S. Cl. CPC A61N 5/022; A61N 5/02

(57) ABSTRACT

A method of treating or preventing an HPV infection or a disease or condition associated with an HPV infection comprises exposing a subject with an HPV infection to microwave energy, where the microwave energy degrades the HPV E6 protein.

### CROSS REFERENCE TO RELATED APPLICATIONS

The present application is a continuation of U.S. application Ser. No. 16/869,886, filed on May 8, 2020, which application claims the benefits of and priority, under 35 U.S.C. § 119(e), to U.S. Provisional Application Ser. No. 62/846,071, filed on May 10, 2019, entitled “USES OF MICROWAVE ENERGY.” The entire disclosures of the applications listed above are hereby incorporated by reference, in their entireties, for all that they teach and for all purposes.

### FIELD

The present disclosure is generally directed to treatment of cells with electromagnetic radiation and, in particular, toward treating pathogenesis with microwaves.

### BACKGROUND

The p53 protein plays important roles in the regulation of the cell cycle, apoptosis and DNA repair. In particular, p53 expression can help prevent uncontrolled cell growth and is often referred to as a tumour suppressor.

In humans, the p53 protein is encoded by the TP53 gene which is located on the short arm of chromosome 17 (locus 17q13.1).

Certain viral pathogens have evolved pathogenesis mechanisms which interfere with native p53 expression and facilitate pathogenesis leading to disease. In some cases, viral associated cancers are attributed, in part to viral processes which inhibit or suppressed some aspect of the function, activity and/or expression of p53.

Human Papillomavirus (HPV) is a prime example of a pathogen with pathogenic mechanisms that interfere with a cells normal p53 functions. In particular HPV expresses a number of oncoproteins, including E6. The E6 oncoprotein is associated with the events that result in the malignant conversion of cells infected with HPV. It is thought that HPV E6 protein mediates ubiquitination of p53; thereafter both E6 and p53 form a protein complex and this complex is subject to proteasomal degradation. As a consequence, the intracellular function of p53 is lost. The loss of p53 function means that the associated growth-arrest and apoptosis-inducing activities are abrogated—it is this process which is important in the pathogenesis of HPV infections.

HPV infect keratinocytes in the basal layers of stratified epithelia at a variety of anatomical sites and their replicative cycle is intimately linked with the differentiation of the infected cell. In many cases HPV causes lesions that can progress to cervical carcinoma.

### SUMMARY

Microwave energy in a form on non-ionising radiation and its application or administration to living cells and tissues causes no DNA damage. The present disclosure provides methods based upon the use of microwave energy which may be used to treat and prevent infections as well as modulate p53 expression in infected cells.

Embodiments of the present disclosure include a method of treating or preventing an HPV infection or a disease or condition associated with an HPV infection, the method comprising exposing a subject with an HPV infection to microwave energy, where the microwave energy degrades the HPV E6 protein.

Embodiments of the present disclosure include a method of treating a cell, tissue, or lesion comprising HPV, the method comprising exposing the cell, tissue, or lesion to microwave energy, where the microwave energy degrades the HPV E6 protein.

Embodiments of the present disclosure include a method of decontaminating a cell or tissue of HPV, the method comprising exposing the cell or tissue to microwave energy, where the microwave energy degrades the HPV E6 protein.

Embodiments of the present disclosure include a method of treating or preventing cervical cancer, the method comprising exposing at least a part of a cervix or a cancerous or pre-cancerous lesion thereof to microwave energy, where the microwave energy creates at least a state of localised hyperthermia in a cell or tissue of the cervix, cancerous lesion or precancerous lesion thereof to degrade the HPV E6 protein.

Embodiments of the present disclosure include an apparatus for use in a method of modulating p53 expression in a cell, the apparatus comprising a microwave source for providing microwave energy and means for administering or delivering the microwave energy to a subject to be treated.

### BRIEF DESCRIPTION OF THE DRAWINGS

FIG. 1 shows microwave energy inducing apoptosis in cervical cancer cells. Staining shows increased Caspase 3 expression adjacent the microwave treatment area.

FIG. 2 shows microwave energy inhibiting cellular proliferation in cervical cancer cells. Panels show reduced Ki67 staining in tissue outside the microwave treatment area.

FIG. 3 shows microwave energy re-introducing p53 expression.

FIG. 4 shows microwave energy reducing the expression of HPV E6.

FIG. 5 shows additional data showing microwave energy reducing the expression of HPV E6.

### DETAILED DESCRIPTION

Exemplary aspects of the present disclosure are described in connection with methods for applying microwaves to cells and/or tissues. However, it is to be understood that the disclosure is not limited in its application to the details of construction and arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The present disclosure is based on the finding that microwave energy may be used to create a state of hyperthermia in a cell or tissue and that this can affect the expression, function and/or activity of certain pathogenicity factors (including, for example the HPV E6 protein) and aspects of the function, activity and/or expression of p53 expression in the cell or tissue.

Based on this fining, the disclosure provides a method of treating or preventing an HPV infection or a disease or condition associated with an HPV infection, the method comprising exposing a subject with an HPV infection to microwave energy, wherein the microwave energy degrades the HPV E6 protein.

Without wishing to be bound by theory, it is suggested that the administration of microwave energy to a subject can create at least a localised (discrete or restricted) state of hyperthermia within the subject and specifically within certain tissues or organs. This state of hyperthermia causes a degradation of the HPV E6 protein. One of skill will appreciate that the HPV E6 protein mediates ubiquitination of p53; thereafter both E6 and p53 form a protein complex and this complex is subject to proteasomal degradation. As a consequence, the intracellular function of p53 is lost. The loss of p53 function means that the associated growth-arrest and apoptosis-inducing activities are abrogated—this is important in the pathogenesis of HPV infections.

Microwave induced hyperthermia interferes with this process as E6 is degraded via both the lysosomal and proteasomal pathways, and this permits p53 to escape proteasomal degradation; in turn, p53 is accumulated within the cell and can become activated, causing G2-phase arrest and p53-dependent apoptosis.

Thus, by degrading the HPV E6 protein, the administration of microwave energy has the effect of restoring p53 function and expression in the host cell.

Accordingly, the method described herein may be administered (or applied) to any subject with and HPV infection and/or any subject predisposed or susceptible to an HPV infection.

The methods described herein may be used in the treatment or prevention of cancer, including cervical cancer.

Additionally or alternatively, the above-mentioned method may be administered or applied to a cell, a tissue (it should be noted that the term “tissue” includes whole or partial organs and/or structures like bones, cartilage and the like) and/or a lesion which comprises an HPV virus. For example, the microwave energy may be administered to a cell, tissue or lesion which exhibits symptoms or signs of an HPV infection. The microwave energy may be administered to a wart, a genital wart, a verruca or a tumour (including, for example, a cervical tumour).

A method of this disclosure may be applied or administered to a cancerous and/or pre-cancerous cervical lesion.

It should be noted that in contrast to prior art methods, one non-limiting advantage of the instant disclosure is that one of the provided methods unexpectedly does not require an active or primed systemic or local immune system. For example, the methods disclosed herein do not require the presence of functional aspects of the local immune system, including for example, heat shock proteins and/or signaling as provided by dendritic and keratinocyte cells etc.

In view of the above, this disclosure may not only find a number of in vivo applications (including methods of treating or preventing HPV infections) but also in vitro applications. For example, a method of one of the embodiments of this disclosure may be applied to an isolated cell, or tissue which cell or tissue comprises HPV and the method is applied to degrade the HPV E6 protein. A method of this type may help eradicate an HPV infection (via E6 protein degradation) from an isolated (ex vivo) cell (including a cell population or culture) or tissue. For example, an isolated cell or tissue to be subjected to a method of this disclosure may be a donor cell or tissue (including whole or partial organs/structures as defined above), for transplant or use in a recipient patient. Thus the methods described herein may be used to decontaminate donor cells and tissues of HPV.

In the disclosed methods of treating or preventing an HPV infection and decontaminating isolated cells and tissues of HPV, the microwave energy may be applied at a dose (i.e. power/energy level and/or time) effective to create a state of hyperthermia.

The state of hyperthermia may be a localised state of hyperthermia.

The term “hyperthermia” refers to the elevation of temperature within a cell or tissue beyond the normal or baseline temperature. For example, in a human, a state of hyperthermia may be defined as a temperature within a human cell or tissue of above 37° C. For example, temperatures of 38° C., 39° C., 40° C., 41° C., 42° C., 43° C., 44° C. or 45° C. may be regarded as defining a state of hyperthermia.

The term “localised hyperthermia” means a state of hyperthermia which is restricted to a specific cell, cells or tissue. For example a state of localised hyperthermia may refer to an elevated temperature which occurs only in a specific cell or cells or only in a specific tissue. A state of hyperthermia induced by a microwave dose defined herein may be confined or restricted to a particular cell type or tissue (including lesions or tumours). For example, the specific cell(s) or tissue may be (a) diseased cell(s) or tissue (including a lesion or tumour). The term “localised hyperthermia” may also extend to a state of hyperthermia in the vicinity of, or near a diseased cell, tissue or lesion.

A diseased cell, tissue, structure, function or lesion may be any cell, tissue, structure, organ or lesion exhibiting one or more symptoms associated with a disease or condition associated with HPV and/or comprising an HPV particle.

In view of the above, disclosed herein is, among other things, a method of treating or preventing an HPV infection or a disease or condition associated with an HPV infection, the method comprising exposing a cell or tissue comprising an HPV E6 protein to microwave energy, wherein the microwave energy degrades the HPV E6 protein.

Another embodiment of the present disclosure includes a method of treating or preventing an HPV infection or a disease or condition associated with an HPV infection, the method comprising exposing a cell or tissue comprising an HPV E6 protein to microwave energy, wherein the microwave energy raises the temperature of the cell or tissue to a temperature selected from a group comprising: (i) 38° C.-45° C.; (ii) 39° C.-44° C.; (iii) 40° C.-43° C.; (iv) 41° C.-42° C.; (v) 38° C.; (vi) 39° C.; (vii) 40° C.; (viii) 41° C.; (ix) 42° C.; (x) 43° C.; (xi) 44° C.; or (xii) 45° C.; wherein the raised temperature degrades the HPV E6 protein.

Embodiments of the present disclosure also include a method of treating or preventing cervical cancer, the method comprising exposing at least a part of a cervix or a cancerous or pre-cancerous lesion thereof to microwave energy, wherein the microwave energy creates at least a state of localised hyperthermia in a cell or tissue of the cervix, cancerous lesion or precancerous lesion thereof.

Embodiments of the present disclosure also include a method of decontaminating a tissue or cell, for example an isolated tissue or cell, of HPV, the method comprising exposing a cell or tissue to be decontaminated, for example an isolated cell or tissue to be decontaminated, to microwave energy, and creating a state of hyperthermia within the cell or tissue, for example the isolated cell or tissue.

As previously detailed, creating a state of hyperthermia within a cell or tissue (for example an isolated cell or tissue) may comprise elevating the temperature of the cell or tissue to a temperature selected from the group of temperatures comprising (i)-(xii) listed above.

In addition or alternatively to the above, this disclosure provides a method of modulating p53 expression in a cell, the method comprising exposing the cell to microwave energy.

The cell may be exposed to a p53 modulating amount of microwave energy.

The cell may be exposed to microwave energy at a power and for a time sufficient to modulate p53 expression in the cell.

A “p53 modulating amount of microwave energy” or microwave energy “at a power and for a time sufficient to modulate p53 expression in the cell” may comprise microwave energy which creates a state of hyperthermia (as defined above with reference to the methods of treating/preventing an HPV infection and or decontaminating a tissue or cell of HPV) within the cell.

The expression of p53 within a cell exposed to microwave energy may be modulated such that, relative to a cell (for example the same cell) not exposed to microwave energy, the level of p53 expression is increased.

A cell to be exposed to microwave energy may exhibit a baseline level of p53 expression. That baseline level of p53 expression may be modulated (for example increased) by exposing the cell to microwave energy. One of skill will understand that dependent on the cell type, the cell cycle, disease status and the like, the (baseline) level of p53 expression (without microwave treatment) may be variable. For example, a particular cell may, for one reason or another, exhibit a low or a reduced level of p53 expression—in such cases, one or more embodiments of the present disclosure may be used to increase the level of p53 expression in such a cell.

Any modulation in p53 expression occurring as a consequence of exposure to microwave energy may be determined by comparison to the level of p53 expression in a cell of the same type, status (for example same disease and/or cell cycle status) and baseline level of p53 expression and not exposed to microwave energy.

In a cell to be subjected to a method of this disclosure, the gene responsible for p53 expression may be suppressed or inhibited. Additionally or alternatively, expressed p53 may be rendered inactive and/or at least some aspect of its function and/or activity suppressed by some means. By way of non-limiting example, a cell infected by a pathogen may comprise proteins (produced by the pathogen) which target and/or bind to host cell p53 rendering it inactive and/or marking it for internal destruction by cell-based proteolysis systems.

Accordingly, the methods of this disclosure may be used in infected or transformed cells to re-introduce, re-establish, stimulate and/or increase p53 expression. One of skill will appreciate that any increase in p53 expression (as might occur following exposure to microwave energy) might overcome any suppression of p53 expression, function and/or activity by a pathogen. In other words and without wishing to be bound by any particular theory, microwave energy may be used to facilitate p53 expression within a cell, such expression overcomes the problems associated with the blocking of native or wildtype levels of p53 by a pathogen.

“p53” may otherwise be known as tumour protein p53. In humans, the p53 protein is encoded by the TP53 gene which is located on the short arm of chromosome 17 (locus 17q13.1). The p53 protein plays important roles in the regulation of the cell cycle, apoptosis and DNA repair. In particular, p53 expression can help prevent uncontrolled cell growth and is often referred to as a tumour suppressor.

As such, and again without wishing to be bound by theory, the finding that microwave energy can be used to modulate p53 expression in a cell is useful in the treatment of certain diseases and/or conditions which are characterised by aberrant p53 expression. Such diseases and/or conditions may include, for example cell proliferation disorders, cancers and the like.

As such, the methods described herein may find particular application in the treatment and/or prevention of a disease or condition selected from the group consisting of: (i) cell proliferation disorders; and (ii) cancers.

The term cancer may include, for example, cancers that are associated with aberrant or modulated p53 expression.

The term cancer may include, cancers that are associated with or caused by a pathogen, for example a virus.

The methods of this disclosure may be applied to cells or tissues (including lesions, organs or structures) that are infected (or affected) by pathogens or affected by a disease or condition, that modulates p53 expression in cells. Where the disease or condition is a cancer, the cancer may be associated with a pathogen that modulates host cell p53 expression.

By way of non-limiting example, the term cancer may embrace a cancer associated with or caused by human papilloma virus (HPV), Epstein-Barr virus (EBV), Hepatitis B Virus (HBV), Hepatitis C Virus (HCV), Human Immunodeficiency Virus (HIV), Human Herpes Virus (HHV including HHV8), Human T-lymphotrophic virus-1 (HTLV-1) and the like.

The cell to be subjected to a method of this disclosure, may be any type of cell-including, for example, a stem cell (an embryonic stem cell or an adult stem cell), a blood cell (for example a white blood cell (including granulocytes and/or agranulocytes (monocytes and lymphocytes))), a nerve cell, a muscle cell (for example a skeletal muscle cell, a cardiac muscle cell or a smooth muscle cell), a cartilage cell, a bone cell, a skin cell (for example a keratinocyte, a melanocyte, merkel cells, Langerhans cells and the like), an endothelial cell and/or an epithelial cell, a muscle cell, a cartilage cell, a nerve cell, a blood cell (for example a white blood cell and/or a red blood cell).

The cell may be comprised within an organ, a tissue and/or a structure. Thus, the methods of this disclosure may be applied to a tissue and/or an organ or structure.

The disclosed methods may be used to induce p53 expression in one or more different cell types.

The cell may be a diseased cell. A diseased cell may be any cell (including any cell type listed above) exhibiting traits or symptoms consistent with an infection. For example, the cell may comprise a bacterial, viral or fungal infection. The cell may be infected by one or more viruses selected from a group comprising: (i) Human Papilloma Virus (HPV); (ii) Epstein-Barr virus (EBV); (iii) Hepatitis B Virus (HBV); (iv) Hepatitis C Virus (HCV); (vi) Human Immunodeficiency Virus (HIV); (vii) Human Herpes Virus (HHV including HHV8); and (viii) Human T-lymphotrophic virus-1 (HTLV-1).

The cell may aberrantly grow, differentiate and/or develop.

The cell may be a tumour or cancer cell.

The cell may be an in vivo cell.

The cell may be an in vitro cell.

The cell may be comprised within an in vivo or in vitro tissue, organ or structure.

The cell may exhibit aberrant (for example reduced) p53 expression. For example, the gene(s) responsible for or associated with p53 expression in the cell may be suppressed or inhibited. Additionally or alternatively, expressed p53 may be suppressed, inhibited or degraded perhaps through the actions of one or more pathogens. As such, the methods of this disclosure may be used to re-introduce, re-establish, stimulate and/or increase p53 expression in a cell in which p53 expression is suppressed or reduced.

Where the method is applied to a tissue (organ or structure) and/or to a population of one or more different cell types, the method may be effective to simultaneously induce p53 expression in a number of the cells within the tissue and/or population.

Microwave energy may have a frequency of between about 500 Megahertz (MHz) and about 200 Gigahertz (GHz). For example, in some embodiments, the frequency of the microwave energy may range from between about 900 MHz and about 100 GHz. In particular, the frequency of the microwave energy may range from about 5 GHz to about 15 GHz and, in one embodiment, may have a frequency of 8 GHz.

It should be understood that embodiments of the present disclosure may use microwave energy having a single frequency, and/or may implement microwave energy across a range of frequencies.

Further, the embodiments of the present disclosure may use microwave energy at a single power, or alternatively microwave energy at a range of different powers. For example, some embodiments may expose a cell to microwave energy at a particular power rating (for example the microwave energy may be used at about 1 Watt (W) of power, 2 W, 3 W, 4 W, 5 W, 6 W, 7 W, 8 W, 9 W or about 10 W).

The microwave energy may be applied at an energy of anywhere between about 1 Joule (J) of energy and about 500J, for example, about 5J to about 200J. The microwave energy may be used at about 5J, about 10J, about 50J, about 100J or about 200J.

When used in any method of this disclosure, the microwave energy may be applied for any suitable duration of time. The microwave energy may be applied for anywhere between about 0.1 seconds(s) and about 60 seconds (1 minute). The microwave energy may be applied for about 1s, about 5s, about 10s, about 20s or about 30s. The microwave energy may be applied as multiple (repeat) bursts or pulses of the same or different duration and/or of (or at) the same or different energy/power level. The number of bursts, the duration of the bursts or pulses, and/or the energy/power level at which the bursts are applied are in no way limited to the above, and various combinations may be used. Each applied microwave energy burst/pulse may last for the same or a different duration. An applied amount of microwave energy may be described as a “microwave energy dose.”

The applied or administered microwave energy dose may (as described above) create a state of hyperthermia within a cell or tissue to be treated or administered a method of this disclosure.

Thus, a method of: (a) treating or preventing an HPV infection; (b) decontaminating a cell or tissue (for example an isolated cell or tissue) of HPV; and/or (c) modulating p53 expression in a cell; may comprise exposing the cell to microwave energy at a dose (i.e. power/energy level and time) effective to modulate p53 expression in the cell or tissue.

Suitable doses of microwave energy may include, for example: 1) 5 W for 30 seconds; 2) 10 W for 10 seconds; or 3) 10 W for 2 seconds.

Each dose may be repeated one or more times.

Any given dose may be administered multiple times, with each administration being separated by a predetermined time interval (for example any time between about 1 second and 1 minute). The dose may be repeated over any predetermined treatment period such. For example, the treatment period may be a short 10-30 second period or a longer period of about 1 minute, 5 minutes, 10 minutes, 15 minutes, 20 minutes or longer. By way of non-limiting example, microwave energy at a selected power (for example 10W or 5 W) may be administered for a predetermined period (for example 1 or 2 seconds) and repeated at regular intervals (for example every 10 seconds) over a predetermined treatment period (for example a total treatment period of about 5 minutes).

Some embodiments may exploit more than one dose. For example a cell, a tissue/organ or subject may be exposed to different microwave doses over a particular treatment period.

A subject may be delivered one or more microwave energy doses over a predetermined period of time. For example, a subject may be administered a single dose on 1 day or multiple doses over the same day, each dose being separated by a non-dosing period (or interval). Additionally or alternatively, a subject may be administered other doses on subsequent days. A treatment (comprising one or more microwave energy dose) may last a day, multiple days or one or more weeks months or years.

One of skill will understand that depending on the cell type or status (for example the cell cycle or disease status), tissue/organ/structure type and/or status (for example disease status) or subject type or status (for example disease status), the microwave energy dose required to effectively modulate p53 expression may vary according to empirical evidence and/or preference.

The disclosure further provides an apparatus for use in a method of modulating (for example increasing) p53 expression in a cell, the apparatus comprising a microwave source for providing microwave energy and means for administering or delivering the microwave energy to a subject to be treated. The apparatus may be used in any of the methods described herein, including, for example, in any of the therapeutic methods of this disclosure.

The apparatus may comprise means for controlling at least one property of the microwave energy produced by the microwave source. For example, the apparatus may enable the user to control or modulate the output power of the microwave energy, the frequency, the wavelength and/or the amplitude of the microwave energy. The means for controlling the microwave energy may be integral with the apparatus or separately formed and connectable thereto.

In one embodiment, the microwave energy source may produce microwave energy at a single frequency and/or microwave energy across a range of frequencies. As stated, a user may be able to select or set a particular microwave or microwaves to be produced by the apparatus and/or the properties of the microwave(s) produced.

The apparatus may further permit monitoring of the microwave energy produced or generated by the microwave source. For example, the apparatus may include a display indicating one or more properties of the microwave energy.

The apparatus may further comprise a device or component for administering or delivering the microwave energy to a cell or to a tissue, structure or organ. The device may comprise an applicator formed, adapted and/or configured to deliver or administer microwave energy to cell and/or to a particular tissue, organ and/or structure.

The inventor has discovered that the dielectric properties of a cell, tissue, organ or structure affected by a disease and/or condition may vary with respect to normal, healthy, tissue (i.e., tissue not affected by the (or a) disease and/or condition). As such, the device for delivering microwave energy may electrically match the range of epsilon relative values of the cell, tissue, structure or organ to be subject to a method of embodiments of the present disclosure and/or affected by a disease and/or condition. In this way, it is possible to ensure efficient delivery of the microwave energy to the cell, tissue, structure or organ.

Advantageously, one embodiment of an apparatus (or device part thereof) for delivering the microwave energy to a subject may comprise a component or part for contact with a cell, tissue, organ, structure or subject to be administered microwave energy according to a method of this disclosure. The part or component for contact may be removable such that it can be discarded or sterilised after use. In one embodiment, the apparatus described herein (or device part thereof) may comprise a single application element or a hand piece which accepts a removable tip which can either be a single use, disposable component or a reusable component intended to be sterilized between uses. Advantageously, the part or component for contact with the cell, tissue, organ, structure of subject to be treated may comprise a re-use mitigation function to prevent accidental or attempted re-use.

The part or component for contact with the subjected to be treated may be shaped, formed or adapted so as to be compatible with a particular internal or external body part, organ, structure or surface. For example, the part or component may comprise a domed, curved or enclosing surface, compatible with the physical properties or profile of an internal or external body part, tissue, organ, structure or surface.

An apparatus of this disclosure may be suitable for the application of microwave energy to the cervix or to a pre-cancerous or cancerous lesion thereof. For example, the apparatus may include a probe, hand-piece or wand which may be used to deliver microwave energy to the cervix and/or a tissue, part or structure thereof.

The part (e.g. probe, hand-piece or wand) for delivering the microwave energy to a subject may be connected to the microwave source via a flexible cable. In one embodiment the means for delivering the microwave energy to a subject (i.e. the applicator) may be connected to the microwave source via a flexible cable with locking connections having both microwave and signal data cables and may be reversible to enable connection to either port.

In one embodiment, the present disclosure provides an apparatus for delivering microwave energy to infected tissue the apparatus comprising: a microwave source for providing microwave energy, connectable to a system controller for controlling at least one property of the microwave radiation provided by the microwave source; and a monitoring system for monitoring the delivery of energy and an applicator means, for example an applicator device, for delivering microwave energy, wherein: the applicator is configured to deliver precise amounts of microwave energy provided by the source at a single frequency or across a range of frequencies.

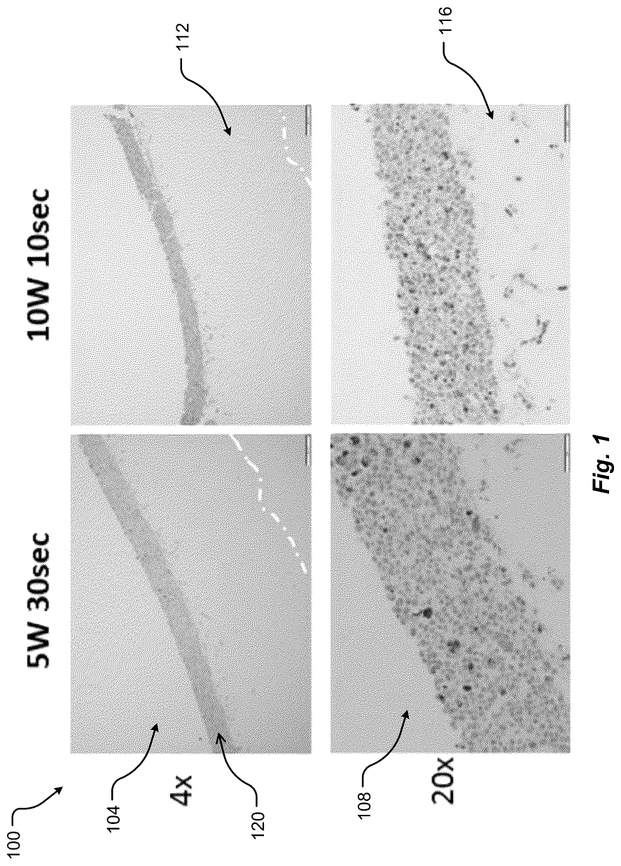

FIG. 1 illustrates a staining experiment 100 in accordance with embodiments of the present disclosure. In particular, the experimental results shown in staining experiment 100 include a frame 104 that illustrates results of a microwave device (not shown) providing energy at a power of 5 Watts (W) for 30 seconds(s) at a 4× magnification; and a frame 108 that shows the same power and time exposure at a 20× magnification. Additionally, a frame 112 shows results of the microwave providing a power of 10 W for 10s at a 4× magnification. A frame 116 demonstrates the same results of the frame 112, but at a 20× magnification.

FIG. 1 indicates apoptosis in cervical cancer cells 120. In particular, caspase 3 production appears greater in the cells adjacent to the microwave treatment area.

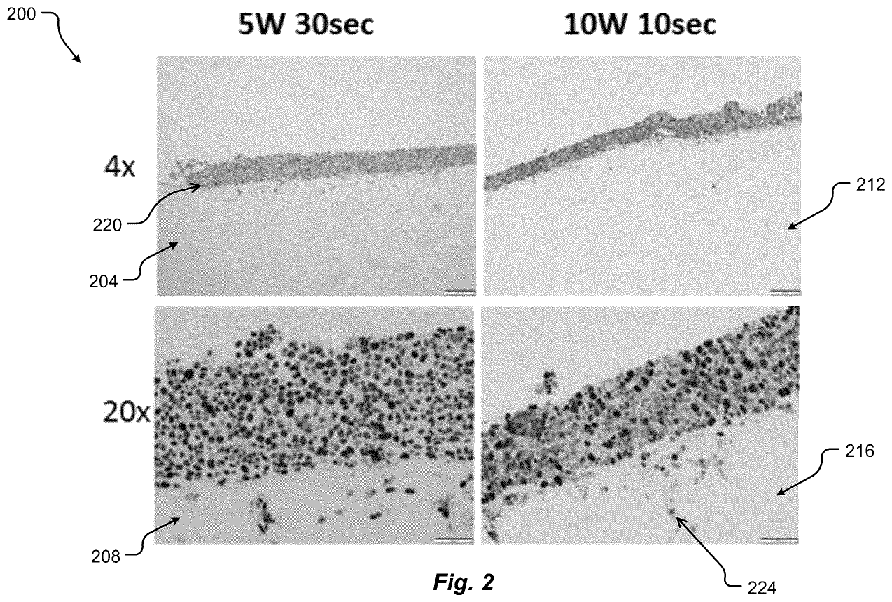

FIG. 2 shows a staining experiment 200 in accordance with embodiments of the present disclosure. In particular, the staining experiment 200 shows a frame 204 detailing the microwave device providing energy at the 5 W power for 30s at a 4× magnification, with a frame 208 detailing the same at 20× magnification. A frame 212 shows the results of a microwave device applying the power 10 W for 10s, and a frame 216 showing the same microwave power and time, but at a 20× magnification.

FIG. 2 shows inhibition of cervical cancer cells 220. For instance, the staining of the staining experiment 200 indicates that the cells outside the microwave treatment area 224 exhibit reduced Ki67 activity, indicating the cells are not dividing.

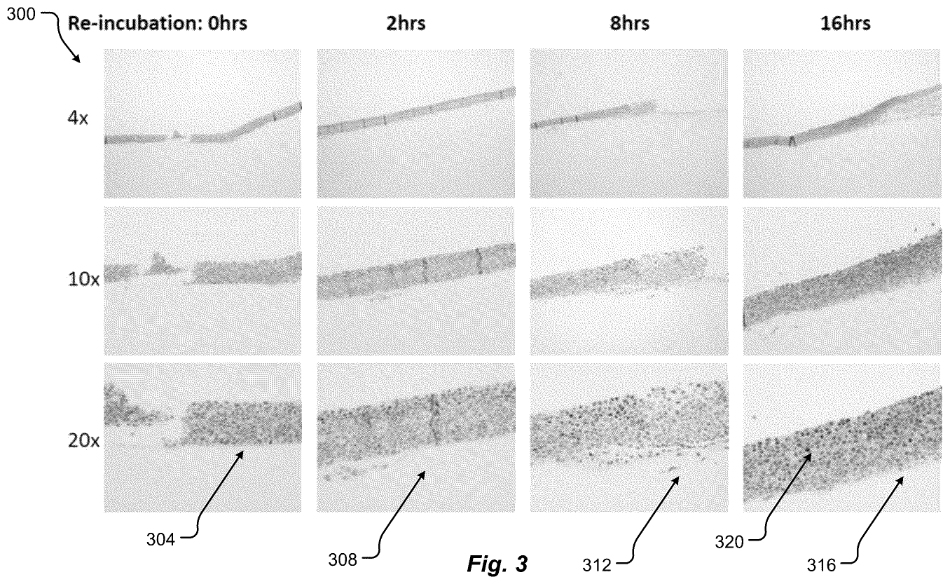

FIG. 3 shows the ability for the microwave energy device to re-introduce p53 expression in cells. In FIG. 3, re-incubation of cells after microwave energy treatment was monitored over 0 hours, 2 hours, 8 hours, and 16 hours from microwave energy application, as indicated by frames 304, 308, 312, and 316, respectively. The progression of frames 304, 308, 312, and 316 indicate a revival in the amount of p53 in the cells. As previously noted, this indicates that the p53 may have formed a complex with the HPV E6 protein, and was then separated from the HPV E6 protein, indicating the HPV E6 protein has been degraded, denatured, or otherwise inhibited from forming a complex with the p53. As noted by dark coloration 320 in frame 316, at 16 hours the concentration of p53 has greatly increased, indicating the microwave treatment caused an increase of p53, which may be detrimental to the HPV.

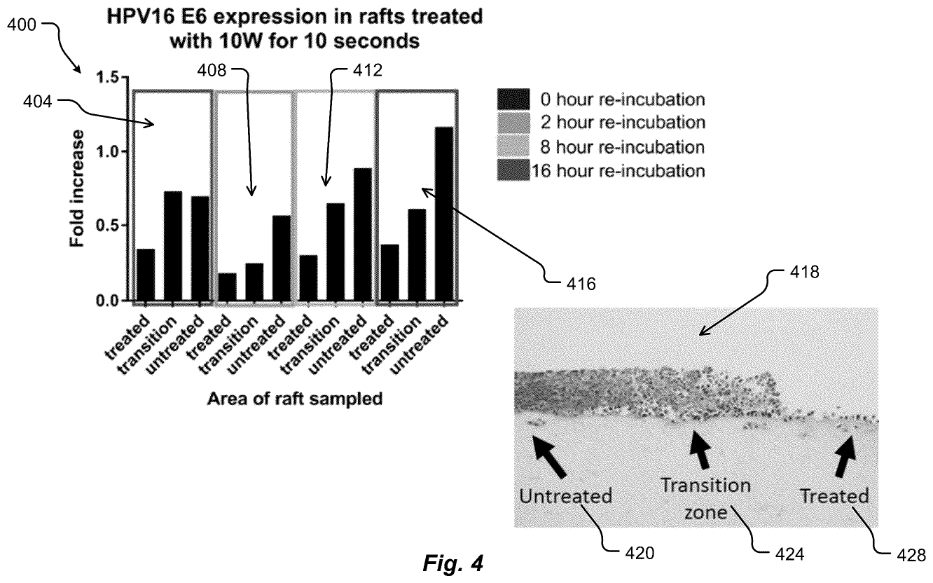

FIG. 4 provides data 400 relating to the effectiveness of reducing the expression of HPV E6. For instance, an experiment was conducted by applying microwave energy at a power of 10 W for 10 s on cells. The cells were then re-incubated. A subsequent area of the cells was sampled at 0 hours (i.e., immediately following the microwave energy application), 2 hours, 8 hours, and 16 hours after the microwave energy application for expression of the HPV16 protein, as indicated by data 404, 408, 412, and 416, respectively. The data 400 compare the fold increase in a zone treated with the microwave energy, a zone absent treatment from the microwave energy, and a transition zone therebetween. As the data 400 indicate, the treated zone has a significantly lower fold increase in the HPV16 protein than the untreated area. This may indicate that the microwave energy is effective at suppressing the HPV16 protein expression.

FIG. 4 also shows a frame 418 depicting an area of the raft of cells that was sampled during the experiment. As indicated by untreated zone 420 (i.e., a zone where the microwave energy was not applied), the HPV16 cells had a much higher protein expression than a treated zone 428 (i.e., a zone where the microwave energy was applied), with a transition zone 424 showing greater protein expression than the treated zone 428, but less protein expression than the untreated zone 420.

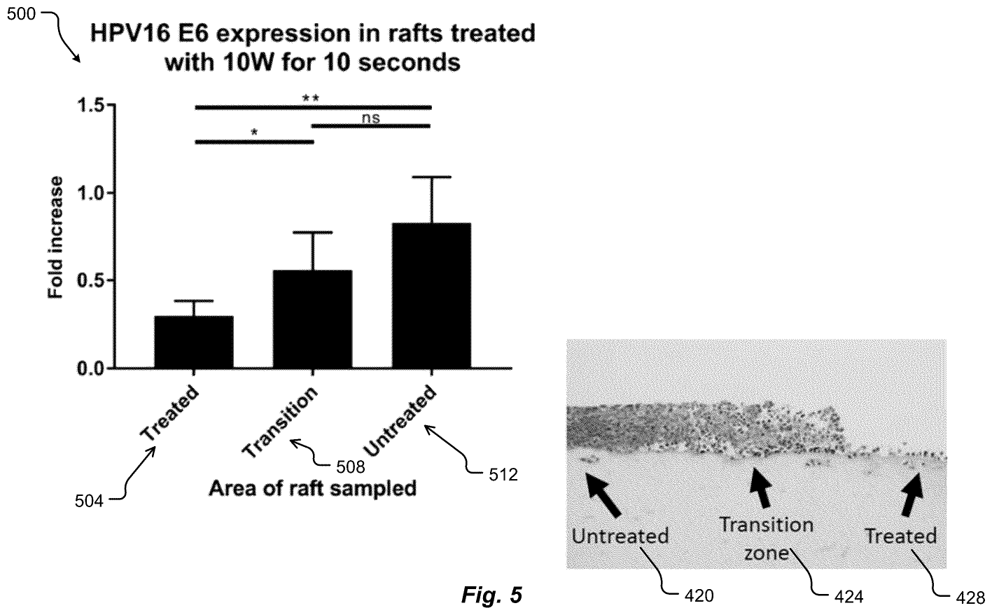

FIG. 5 shows additional data 500 showing the suppression of HPV16 E6 protein expression by microwave energy treatment. In addition to showing the frame 418, the additional data show a graph showing the averaged fold increases for each of the untreated, transition, and treated zones 420, 424, 428 with untreated data 512, transition data 508, and treated data 504, respectively. Each of the untreated data 512, transition data 508, and treated data 504 indicate the average fold increases by averaging the fold increases across the 4 time periods during which sample data was collected. In other words, the treated data 504 is an average of the fold increase numbers collected at the 0 hour, 2 hour, 8 hour, and 16 hour marks. The same averages were also collected for the transition data 508 and the untreated data 512. As noted by the additional data 500, the treated data 504 had a sub 0.5 fold increase even when considering the possible statistical error. In contrast, the untreated data 512 illustrates a much higher fold increase, including the possibility of a greater than 1.0 fold increase when considering the statistical error. This difference indicates that the microwave energy treatment may contribute to increased suppression of the HPV16 E6 protein expression.

What is claimed is:

1. A method of treating or preventing an HPV infection or a disease or condition associated with an HPV infection, the method comprising:
exposing a subject with an HPV infection to microwave energy, wherein the microwave energy is applied at either 5 Watts (W) for 30s, 10 W for 10s, or 10 W for 2s, and wherein the microwave energy degrades HPV E6 protein.

2. The method of claim 1, wherein degrading the HPV E6 protein restores p53 function and expression in a host cell.

3. The method of claim 1, wherein the subject is predisposed or susceptible to an HPV infection.

4. The method of claim 1, wherein the method is for the treatment or prevention of cervical cancer.

5. A method of treating a cell, tissue, or lesion comprising HPV, the method comprising:
exposing the cell, tissue, or lesion to microwave energy, wherein the microwave energy is applied at either 5 Watts (W) for 30s, 10 W for 10s, or 10 W for 2s, and wherein the microwave energy degrades HPV E6 protein.

6. The method of claim 5, wherein the lesion is cancerous or precancerous.

7. The method of claim 5, wherein the lesion is a cancerous or precancerous cervical lesion.

8. The method of claim 5, wherein the lesion comprises a wart, a genital wart, a verruca, a tumour, or a cervical tumour.

9. The method of claim 5, wherein the cell or tissue is an isolated cell or tissue.

10. The method of claim 5, wherein the cell or tissue is a cell or tissue for donation.

11. A method of decontaminating a cell or tissue of HPV, the method comprising:
exposing the cell or tissue to microwave energy, wherein the microwave energy is applied at either 5 Watts (W) for 30s, 10 W for 10s, or 10 W for 2s, and wherein the microwave energy degrades HPV E6 protein.

12. The method of claim 11, wherein the cell or tissue is a cell or tissue for donation.

13. The method of claim 11, wherein the microwave energy raises the temperature of the cell or tissue to a temperature of about 38° C.-45° C.

14. The method of claim 1, wherein the microwave energy has a frequency of between about 500 Megahertz (MHz) and about 200 Gigahertz (GHz), between about 900 MHz and about 100 GHz, between about 5 GHz and about 15 GHz, or has a frequency of about 8 GHz.

15. The method of claim 1, wherein the microwave energy is applied as multiple bursts or pulses of a same or different duration, and wherein the microwave energy is applied at a same or different energy and power level.

16. The method of claim 5, wherein the microwave energy has a frequency of between about 500 Megahertz (MHz) and about 200 Gigahertz (GHz), between about 900 MHz and about 100 GHz, between about 5 GHz and about 15 GHz, or has a frequency of about 8 GHz.

17. The method of claim 5, wherein the microwave energy is applied as multiple bursts or pulses of a same or different duration, and wherein the microwave energy is applied at a same or different energy and power level.

18. The method of claim 11, wherein the microwave energy has a frequency of between about 500 Megahertz (MHz) and about 200 GHz Gigahertz (GHz), between about 900 MHz and about 100 GHz, between about 5 GHz to about 15 GHz, or has a frequency of about 8 GHz.

19. The method of claim 11, wherein the microwave energy is applied as multiple bursts or pulses of a same or different duration, and wherein the microwave energy is applied at a same or different energy and power level.
